(19) United States
(12) Patent Application Publication
(10) Pub. No.: US 2022/0330886 A1
(43) Pub. Date: Oct. 20, 2022

(54) SMART WOUND MANAGEMENT SYSTEM

(71) Applicant: NANOWEAR INC., Brooklyn, NY (US)

(72) Inventors: Vijay Varadan, State College, PA (US); Mouli Ramasamy, State College, PA (US); Pratyush Rai, State College, PA (US); Prashanth Shyam Kumar, State College, PA (US); Venkatesh Varadan, Brooklyn, PA (US)

(73) Assignee: NANOWEAR INC., Brooklyn, NY (US)

(21) Appl. No.: 17/721,025

(22) Filed: Apr. 14, 2022

Related U.S. Application Data
(60) Provisional application No. 63/174,721, filed on Apr. 14, 2021.

(51) Int. Cl. A61B 5/00; A61F 13/00; A61F 13/02; A61B 5/145; A61B 5/0531; A61B 5/01; G16H 20/13; G16H 40/67

(52) U.S. Cl. CPC A61B 5/445; A61F 13/00063; A61F 13/0253; A61B 5/14539; A61B 5/0531; A61B 5/6833; A61B 5/4842; A61B 5/746; A61B 5/0022; A61B 5/7275; A61B 5/01; G16H 20/13; G16H 40/67; A61F 2013/00676; A61B 2562/0215; A61B 2562/0285; A61B 2562/029; A61B 2562/0219

(57) ABSTRACT

The present invention relates to a smart wound management system and to a method of medical treatment and management for wounds.

This application claims priority to U.S. Provisional Application Ser. No. 63/174,721, filed Apr. 14, 2021, entitled Smart Wound Management System, the entire disclosure of which is hereby incorporated by reference.

### FIELD OF INVENTION

The present invention relates to medical treatments and management for wounds, and more specifically to a smart wound management system.

### BACKGROUND

The clinical management of wounds is one of the most difficult challenges to the medical community today.

Chronic non-healing wounds create significant healthcare challenges that affect many people. Although chronic wounds are locked in a persisting inflamed state, they are dynamic and proper therapy requires identifying abnormalities, administering appropriate drugs and growth factors, and modulating the environment's conditions.

Severe wounds can extend fully through soft tissue and involve exposed bone. Severe wounds can also involve undermining of surrounding tissue with significant erosion occurring underneath the outwardly visible wound margins. Significant sinus tract can radiate out from the wound's epicenter with the narrow openings or passageways of the sinus tracts extending underneath the skin in any direction through soft tissue, possibly resulting in dead space with the potential for abscess formation. Wounds with undermining usually have significant drainage, requiring standard dressing changes ranging from daily dressing changes, dressing changes a few times per week or even a few times per day to several times per week, depending on wound drainage rate, healing rate, any infections, etc. These can be some of the most difficult and costly wounds to manage, as patients are often bed ridden and are very difficult to move.

Common types of wound care used widely negative pressure therapy, hyperbaric oxygen therapy, compression therapy and electric pulse therapy.

In negative pressure therapy, a vacuum seal is created using special airtight and watertight foam dressings and gauze are used around the wound to maintain a vacuum seal. A vacuum pump is then used intermittently or continuously over the course of several days to draws out fluids, exudates, and infectious materials from the wound. Negative pressure therapy is used, e.g., in large chronic persistent wounds and acute complicated wounds. While this type of wound therapy treatment is considered effective, there is a risk of hemorrhaging in patients with bleeding issues or when caregivers do not receive proper training on how to operate the device.

In hyperbaric oxygen therapy, patients are placed in a chamber that exposes the body to a 100% oxygen environment at high pressure. Since wounds need oxygen to heal, it is thought that this type of wound therapy can speed the healing process. Hyperbaric oxygen therapy is used for patients with large or full body wounds such as burn victims.

Compression therapy consists of applying a type of elastic device, mainly on the limbs, to exert a controlled pressure on them. By compressing the limbs or other body regions, the medical compression device squeezes the vein walls together, thereby improving overall circulation and supporting blood flow back towards the heart. Compression therapy can also help to reduce swelling and formation of edema Compression stockings work by applying gentle pressure to the ankles and calf muscles which cause vein walls to straighten and improve circulation. Compression therapy is effective for venous ulcers which are wounds on the leg caused by abnormal vein function because it allows the veins to work correctly. Electric pulse therapy is the application of electrical current through electrodes placed on the skin either near or directly on the wound. When the epithelial layers of the skin are injured, the body's naturally-occurring electrical current is disrupted. It is thought that by providing electrical stimulation, a wound's healing process will be accelerated by imitating the body's electrical current. Thus, electrical stimulation is a unique wound treatment option that may help to heal chronic wounds, reduce infection, increase blood flow, and accelerate the wound healing process.

The common denominator is that none of these methods is applicable for all types of wounds as each method is very specific to the type of the wound.

Wound healing is a complex physiological process affected by numerous intrinsic and extrinsic factors. For improved wound care, physiological parameters at the wound site can be monitored. Through evaluation of these parameters, a clinician can determine the wound healing state and whether the wound has developed an infection or would benefit from wound care such as the negative pressure therapy, hyperbaric oxygen therapy, compression therapy or electric pulse therapy mentioned above. Therapeutics such as medications can be administered in response to a change in the physiologic parameters or other therapeutics, such as electrical stimuli, can be administered to enhance tissue regeneration.

### SUMMARY OF THE INVENTION

A new smart sensor system could improve clinical efficacy and e.g., reduce the number of dressing changes required during wound management by monitoring and enhancing the healing rates. Wound healing is a complex physiological process affected by numerous intrinsic and extrinsic factors which can be monitored. For example, for improved wound care, physiological parameters such as pH, temperature, moisture level and microbial activity at the wound site can be monitored.

For a wound to heal, the pH preferably must come down from alkaline (7.15-8.9) to neutral (7) to acidic (4-5.5). A higher pH is correlated to infection and increased microbial activity. An acidic wound dressing is helpful in enhancing the wound healing rate.

Normal skin temperature is between 98.4° F. and 99.4° F. For a healing wound that is not infected, skin temperature will be between 99.4° F. and 100.4° F. and for a wound with an infection, the temperature can be between 100.4° F. and 105.6° F., with higher temperature correlated to more infection and higher microbial activity. For a wound to heal, the skin temperature must come down to normal (98.4-99.4 F).

A wound site can vary between extremely dry, dry, normal, wet and sometime oily, with absolute values or ranges can depend not only on the wound site, wound size and type of wound but also on the location of the sensor, the distance between sensors, the current, and the frequency. The amount of moisture at the wound the site should be close to that of the normal skin for faster healing and therefore it is beneficial to optimize moisture levels within a healing wound. Too little moisture can result in dry wound dressings causing excessive pain on removal and a slowed rate of wound closure. An excessively moist wound can result in tissue edema, maceration, moisture-associated skin damage, and a slower rate of wound resolution.

Wound healing can also be predicted based on the rate of silver nanoparticles depletion, with the rate of silver nanoparticle depletion being proportional to bacterial activity at wound site. Controlled release of silver nanoparticles is achieved based on the pH value measurement and the applied electric potential. Higher rate of silver nanoparticles depletion is correlated to higher bacterial activity and infection. Lower rate of silver nanoparticles depletion is correlated with normal wound healing and less bacterial activity. Absolute values or range can depend on sensor characterization, the amount of silver nanoparticles in sensors during application to determine a baseline value, supporting instrumentation, etc. Depletion of silver nanoparticles can be measured as percentage depletion of the starting silver nanoparticles amount with respect to time.

Wounds also need oxygen to heal. Detection of oxygen level at a wound site can be beneficial, with subsequent infusion of oxygen through e.g., hypobaric oxygen chamber therapy increasing the rate of wound healing.

Biofouling is a major issue in the process of wound management and healing, with more than 90% of chronic wounds affected by biofilms. Biofilms and biofouling are caused by the formation of a thick slimy layer secreted by a community of bacteria which forms a biofilm layer between the wound and the bandage, e.g., an adhesive bandage, such as Band-Aid Adhesive Bandage®, and can affect the functionality of the bandage. The biofilms are not visible to the naked eye and are impenetrable by antibiotics, antiseptics, antimicrobials, or antibodies. Because of these issues, the bacterial colony at the wound site can begin to interact with each other, making the bacteria more resistant to treatment. A conventional bandage is not able to treat this issue, which could continue to worsen due to continuous increase of resistance to treatments by the bacteria. Biofilms can then lead to biofouling, which can degrade the functionality of the bandage.

Antibiofilm agents are not antibiotics, but affect biofilm by disrupting quorum sensing, degrading extracellular polymeric substances, blocking attachments, and many other antibiofilm strategies. Antibiofilm agents allow antibiotics to be more effective. Methods such as laser, ultrasound, acoustic, electrical pulses and currents, material manipulation and alterations, and other methods can physically disrupt biofilms. Altering bacterial migration could be a successful approach for augmenting the natural wound-healing process. These methods have the possibility of targeting biofilm cells and sparing the host cells.

Abnormal wound-site changes can be an early predictor of infection. Besides, on-demand therapy is another requisite for wound management that releases the therapeutic drugs according to wound needs. Compared with the traditional methods for infection methods, the described system provides improved solutions. It is more sensitive, intuitive, and accurate.

It is an object of the present invention to provide a bandage system for wounds capable of collecting data which can then be used for personalized wound care based on demographic, nutrition, type of wound, wound site, underlying conditions, co-morbidities, existing preconditions, and the like. Also, the type of metrics measured will be useful to monitor and treat all kinds of wounds.

It is also an object of the present invention to provide a bandage system for wounds capable of collecting data and also of treating wounds using personalized wound care based on parameters such as demographic, nutrition, type of wound, wound site, underlying conditions, co-morbidities, existing preconditions, and the like. Also, the type of metrics measured will be useful to monitor and treat all kinds of wounds.

It is a further object of the present invention to provide a bandage system for wounds capable of collecting data and/or of treating wounds using personalized wound care wherein the data includes physiologic parameters such as pH, temperature, moisture level and microbial activity.

It is additionally an object of the present invention to provide a method of collecting and assessing data from wounds to be used for personalized wound care. It is a further object of the present invention for the method to include treatment of the wounds based on the assessment of the data.

In accordance with an embodiment of the present invention, an integrated system for assessing a wound is provided.

In accordance with another embodiment of the present invention, an integrated system for assessing and treating a wound is provided.

In accordance with another embodiment of present invention, the system may detect, process and report various wound parameters.

In accordance with another embodiment of present invention, the system may make treatment determinations based on these findings.

In accordance with another embodiment of present invention, the system may detect one or more physiological values from the wound of the patient. In certain preferred embodiments, the data is physiologic data. In preferred embodiments the physiologic data includes pH, temperature, moisture level and microbial activity

In accordance with another embodiment of present invention, the system may compare one or more detected physiological values to predetermined physiological values in order to obtain a comparison result in real time.

In accordance with another embodiment of present invention, the physiological values may be detected by one or more sensors and electronics.

In accordance with another embodiment of present invention, the sensor(s) may be nanostructured working electrode (WE) and counter electrode (CE), and silver-silver chloride reference electrode (RE).

In accordance with another embodiment of present invention, the sensor(s) may be an array connected to an electronics module that acquires sensor signals, sends electrical stimuli and communicates wirelessly to mobile device at programmable time intervals.

In accordance with another embodiment of present invention, the system may compute a composite score for wound healing and to determine the frequency and magnitude of delivery of therapeutics.

In accordance with another embodiment of the invention, the system may include wound treatment components, sensing components for sensing one or more values of one or more physiological parameters of the wound, application components for applying one or more parameters to the wound site, analyzing mechanism for analyzing the values of the one or more physiological parameters so as to obtain an assessment of the wound exudate, and providing means for providing treatment guidelines based on the assessment, in which the wound treatment, sensing, analyzing, and applying means are integrated.

### BRIEF DESCRIPTION OF THE DRAWINGS

FIG. 1(a) illustrates a top view of the bandage system according to embodiments of the present invention, and shows the nanosensor hydrogel stack-cloth-based nanosensor array on a cloth substrate.

FIG. 1(b) illustrates a pattern of inter-digitated electrode (IDT/IDE) pairs that function as a working electrode (WE) and counter electrode (CE).

FIG. 1(c) illustrates a bottom view of the bandage system showing the electromechanical connector.

FIG. 1(d) illustrates a sectional view of the bandage system showing the grid of dedicated connection lines.

FIG. 2 shows cloth based nanosensors with vertically standing nanostructures.

FIGS. 3(a)-3(c) illustrate nanosensor hydrogel stacks with the hydrogel as a conformal coating or placed in form of a pattern that interpenetrates with the pattern of the nanostructured surface. FIG. 3(a) shows a hydrogel film is placed on top of the nanostructures of the nanostructured surface as a conformal coating and FIG. 3(b) shows the hydrogel in the form of a hydrogel pattern that is interspersed with the pattern of the nanostructured surface or nanostructures integrated with the inter-digitated electrode (IDT), which is illustrated in FIG. 3(c).

FIG. 4 shows the data flow of the wound management system.

FIG. 5(a)-(c) show the mechanical design of the electronics module, with the front view (5a), back view (5b) and perspective view (5c), showing Power on/off switch, LED indicators and audio-based alarms, electromechanical connector to connect the electronics module to the bandage.

FIG. 6 shows a block diagram of the electronics module.

FIG. 7 shows a diagram of the steps of code for microprocessor or microcontroller of the electronics module.

FIG. 8 shows a diagram of the software implementation on Signal Acquisition Units as firmware, on Smart Phone as software application and Web Server/Portal.

FIG. 9 shows overall operation of the Smartphone Mobile App.

FIG. 10 shows the sequential commands sent to the electronics module to configure it before the start of a test or recording, with the sequential commands sent from the Mobile App to the Electronics module in order to start the data acquisition for a test and the sequential commands sent from the Smartphone App to the electronics module when the App is restarted after being suspended or shutdown respectively.

FIG. 11 shows the interaction between microcontroller or microprocessor executable code, local storage available in the smart device and cloud services.

FIG. 12 shows a diagram of an exemplary wound management system deployment.

### DETAILED DESCRIPTION OF THE EMBODIMENTS

In accordance with various embodiments of the present invention a system for assessing the wound of a patient is described. The system may detect, process and report various wound parameters. The system may make treatment determinations based on these findings. The system may detect one or more physiological values from the wound of the patient. The system may compare one or more detected physiological values to predetermined physiological values in order to obtain a comparison result in real time. The physiological values may be detected by one or more sensors and electronics. The sensors may be nanostructured working electrode (WE) and counter electrode (CE), and silver-silver chloride reference electrode (RE). The sensor(s) may be an array connected to the electronics module that acquires sensor signals, sends electrical stimuli and communicates wirelessly to mobile device at programmable time intervals. The system may compute a composite score for wound healing and to determine the frequency and magnitude of delivery of therapeutics. Suitable sensors for use in the present invention are described in U.S. patent application Ser. No. 16/916,843, the entire disclosures of which is hereby incorporated by reference.

### Bandage System

Sensor arrays for multiparameter monitoring (e.g., moisture, temperature, pH, silver depletion due to high microbial activity, oxygen content) are described herein. Each parameter is detected by a dedicated set of nanostructured working electrode (WE) and counter electrode (CE), and silver-silver chloride reference electrode (RE). The inter-digitated working electrode (WE) and counter electrode (CE) are nanostructured electrodes. The reference electrode (RE) is a silver-silver chloride printed electrode. The plurality of electrodes is covered with a hydrogel and a thermosensitive hydrogel-drug/buffer formulation. For example, a N-isopropyl acrylamide (NIPA, C6H11NO) based thermosensitive polymer in combination with chitosan for adhesion can be used as hydrogel. A hydrogel conformal film on top of the nanosensor surface or a hydrogel film stacked with the nanosensor can provide moisture for the skin to make it moist for better nanosensor-skin contact. The electrical interface with the skin is still formed by the nanostructures, unlike the gel-based electrodes that rely on the wet chemistry of the salts in the gel to make the electrical interface with the skin.

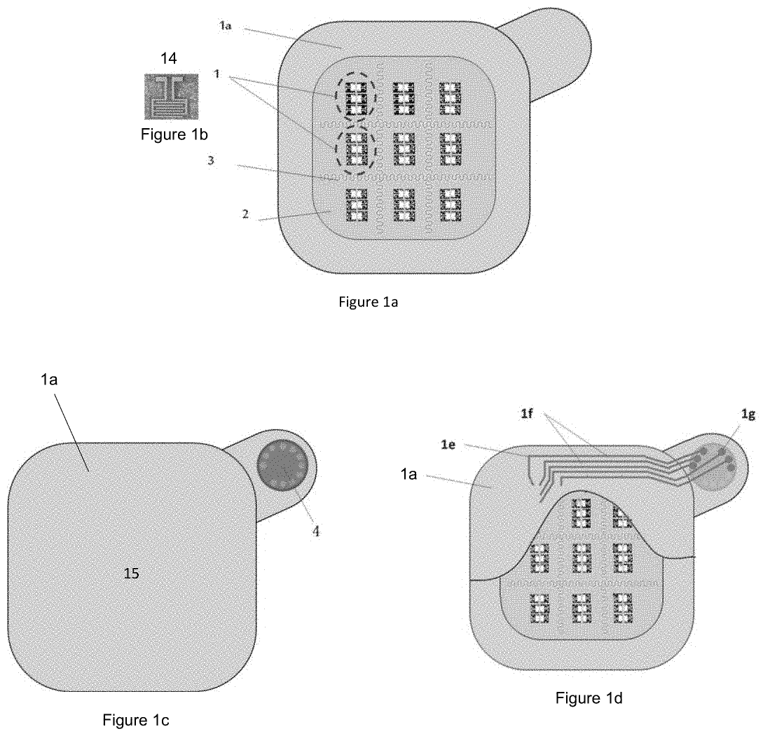

An example of a nanosensor hydrogel stack on a bandage of the present invention is shown in FIGS. 1(a)-1(c). As shown in FIG. 1(a), the bandage is comprised of stack sets, with each stack set comprised of a cloth-based nanosensor with vertically standing polymer nanofibers coated with electrically conductive material to form a conductive nanostructured surface in a predefined pattern on a cloth substrate. An exemplary 3×3 array of three nanosensor stack sets is shown in FIG. 1(a). stack has hydrogel functionalized to make it sensitive to one physiologic parameter such as such as pH, temperature, moisture level and oxygen level of the skin-nanosensor interface. The nanosensor stack has the ability to inject electrical pulses of controlled amplitude and frequency into the skin by passing the pulse between the two nanosensors of the interdigitated electrode pair. The electrical pulses can be used e.g., for bioimpedance to prevent biofilms and biofouling caused by microbial activity. The nanosensor stack also has the ability to detect silver depletion at the surface of the nanosensor by detection of change in impedance between the 2 nanosensors o the interdigitated electrode pair. The nanosensor stack

The cloth substrate is coated with thermosensitive hydrogel. In certain embodiments of the present invention, the thermosensitive hydrogel (2) can be embedded with a drug such as an antibiofilm agent and/or an antibiotic such as amoxicillin-clavulanate, cephalexin, clindamycin, dicloxacillin, doxycycline and trimethoprim-sulfamethoxazole. In certain embodiments, there are heating elements (3) sandwiched between the hydrogel and the cloth substrate or adhesive bandage. The heating elements (3) can be used to trigger drug release.

The cloth based nanosensors with the hydrogel stack is fabricated in patterns of inter-digitated electrode (IDT/IDE) pairs (14) that function as working electrode (WE) and counter electrode (CE) as shown in FIG. 1b. The pair uses a printed silver-silver chloride reference electrode that can be nanostructured for better performance because of high aspect ratio. Each set of WE-CE pair and the sensor electronics (1) work as dedicated sensor for e.g., detection at wound site of a physiological parameter such as oxygen, silver depletion, pH, temperature, moisture level. One dedicated set of WE-CE pair and reference electrode for each of the biomarkers and physiological parameters forms a basic unit of nanosensor array. As depicted in FIG. 1(a), multiple such units of nanosensor array cover the side of the adhesive bandage to enable sensing of the entire wound site.

The layer of nanosensor array has a layer of electrical connection lines that consist of a grid of dedicated connections lines (1f) made of electrically conductive wires or printed conductive lines that are capable of relaying signals of the order of micro-volts or milli-volts and deliver electrical current that between about 1 and about 5 Volts, less than about 1 mA. The electrical connection lines are also connected to a network of printed lines of heat elements (3) that are made of high resistance inks such as inks with carbon fillers or fine wires that have high resistance such as ceramic wires with positive temperature coefficient. The electrical connection line network converges to a connector break out circuit (4). This circuit has a connector mounted on it that is used to connect the electronics module (16) to the bandage system (15). The network of printed lines of heating element (3) is embedded between a layer of thermosensitive hydrogel (2) and the adhesive bandage (1a) and sensor electronics (1). The hydrogel is functionalized with therapeutic chemical compounds such as antiseptic agents, buffer stabilized moisture (or saline). The network of printed lines of heating element (3) is designed to achieve temperatures above the phase transition temperature of the hydrogel to trigger the release of therapeutic chemical compounds.

The nanosensor signal acquisition and triggering heating element network for controlled release of therapeutic chemical compounds is managed by the electronics module (16) that is connected to the nanosensor and heating element network through a connector on the far side (away from the skin) of the bandage.

FIG. 1(c) depicts the opposing side of the bandage of FIG. 1(a) and shows a connector (4) for connecting the bandage to the electronics module. In certain embodiments, the overall bandage foot print is from about 10 to about 15 centimeters in length and from about 10 to about 15 centimeters in width.

FIG. 1(d) is a sectional view of the bandage of FIG. 1(a) and shows the adhesive layer (1a) sandwiched between the adhesive bandage 1(a) and the sensors. Electrical connection lines (1e) consist of a grid of dedicated connections (1f) lines made of electrically conductive wires or printed conductive lines that connect to interconnect (1g).

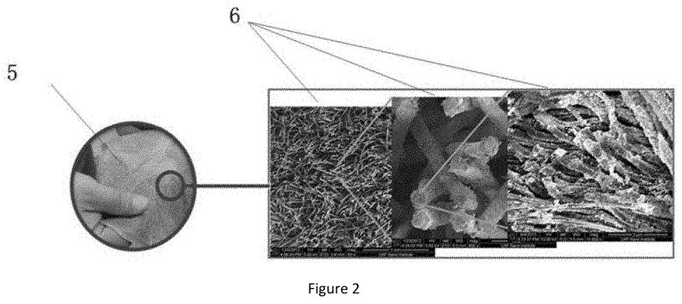

As shown in FIG. 2, the nanosensors can have a conductive nanostructured surface on a cloth substrate 5 that is made of vertically standing pillars (30 microns or less) with nanostructured tips and lateral walls 6 that have nanofilaments with diameter 200 nm or less.

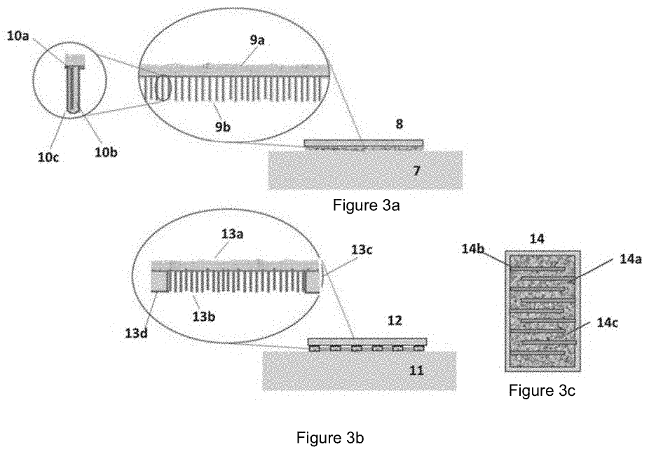

FIGS. 3(a) through 3(c) illustrate wearable nanosensor hydrogel stacks in accordance with the present invention. In both FIG. 3(a) and FIG. 3(b), the stack (8, 12) comprises a cloth-based nanosensor with vertically standing polymer nanofibers coated with electrically conductive material to form a conductive nanostructured surface (9b, 13b) on a cloth substrate (9a, 13a). The nanostructured surface (9b, 13b) also has a film of hydrogel that responds to skin (7, 11) temperature and release moisture.

On the nanosensor hydrogel stack (8) of FIG. 3(a), a hydrogel film (10b) is placed on top of the nanostructures (10a) of the nanostructured surface (9b) as a conformal coating. In the nanosensor hydrogel stack (12) of FIG. 3(b), the hydrogel is in the form of a hydrogel pattern (13c) that interpenetrates or is interspersed with the pattern of the nanostructured surface (13b) or nanostructures integrated with the inter-digitated electrode (IDT) (14) as illustrated in FIG. 3(c). In both the stack (8) and the stack (12), the hydrogel film (10b, 13c) has a cover layer (10c, 13d) on top of it to prevent moisture loss and mechanical stress during wear. The cover layer (10c, 13d) is made of interwoven fibers or non-woven thin film of synthetic polymers such as polyester, butyl rubber, polyolefins etc.

This hydrogel-nanosensor stack forms a good nanosensor-skin contact by introduction of moisture at the nanosensor-skin interface, which is triggered immediately after the stack comes in contact with skin and detects skin temperature. The stack retains moisture at room temperature and can be recharged when it comes in contact with a moisture source.

Interdigitated electrodes (IDE): Interdigitated electrodes (IDE) (14) as shown in FIG. 3(c) are a pair of electrodes with the two electrodes facing each other and have a comb-like pattern. Each tooth or finger of the comb projects out towards the opposite electrode. The tooth or finger/digits reach out alternatively thus forming a pattern similar to two combs inter-locked or interdigitated. This pattern results in a meandering channel (14c) between the two electrodes that is filled with electrolyte material. This results in a substantial channel width increase between the two electrodes, which translates to improved electrochemical interaction between the electrodes. IDEs have been used in electrochemical sensors and electrochemical impedance spectroscopy. The two electrodes are used as working electrode (WE) (14a) and a counter electrode CE (14b). The specific electrochemical reaction happens at the surface of the WE. The electron or charge created by the electrochemical reaction crosses from the solution phase of the electrolyte to the solid phase of the WE and to the external circuit or instrumentation. The CE acts as the source which releases electron or charge to the electrolyte solution, thus completing the electrical circuit.

Thermosensitive hydrogels formulation: Several gels have the characteristic of undergoing a discontinuous volume change upon changes in temperature and are classified as Thermosensitive Hydrogels. This phenomenon is due to the phase transition in the hydrogel at the designated temperature, and at this phase transition temperature (PTT) the swelling ratio of the hydrogel undergoes a sudden change.

There are various hydrogels which exhibit thermosensitive behavior. However, acrylamides and substituted acrylamides show a clearly defined phase transition temperature ranging between about 17 to about 60° C. In particular, N-isopropyl acrylamide (NIPA, C₆H₁₁NO) copolymerized with N,N′ methylene bis acrylamide (BIS, C₇H₁₀N₂O₂) gives the phase transition temperature of 33° C. which is useful for skin contact application.

Hydrogels can be cationic, anionic or neutral in nature and their hydrophilicity is due to the presence of —NH₂, —COOH, —OH, —CONH₂ groups which leads to their swelling in the presence of water.

Hydrogel synthesis: Exemplary hydrogels were synthesized based on N, isopropyl acrylamide (NIPA) as their phase transition temperature was in the range of 33−37° C. N, N′ methylene bis acrylamide (BIS) was used as a crosslinking agent. Ammonium per sulfate (APS) was used as a Redox initiator and Sodium meta bi sulfite (SMBS) as an accelerator. All reactants were dissolved in deionized water and the reaction was carried out under Nitrogen blanket at 5° C. for 12 hrs.

—(C₆H₁₂NO)—_{X1}+—(C₇H₁₀N₂O₂)—_{X2}→^{APA,SMBS}NIPA Hydrogel

The NIPA-BIS hydrogel can be prepared in deionized water by adding 6 to 8 weight % NIPA and 1 to 2 weight % BIS then purging with Nitrogen. After this a mix of 2 to 4 weight % Ammonium per sulfate (initiator) and Sodium Meta BiSulfite (accelerator) in deionized water was added to the above prepared solution and the solution was again purged with Nitrogen. The solution was kept in an airtight container at 5° C. for 12 hours.

Sensor fabrication: The nanosensor fabrication involves steps of embedding polymer nanofibers into a matrix polymer to form a yarn; dissolving the matrix polymer to expose the polymer nanofibers; and coating the polymer nanofibers in a film. The yarn can be a micro denier yarn. The micro denier yarn can have a helical structure. The method can further include a step of imparting an electrostatic charge to the yarn prior to dissolving the matrix polymer. The polymer nanofibers can be made of a polymer material selected from the group consisting of polyethylene terephthalate, polyethylene naphthalate or polybutylene terephthalate. The polymer nanofibers can be made of a polyester. The polymer nanofibers can be made of a polyurethane. The matrix polymer can be made of a material selected from the group consisting of polystyrene, polyvinyl alcohol, ethylene vinyl alcohol, polyacrylamide or poly lactic acid. The matrix polymer can be made of a polyethylene terephthalate modified with sulfonated isocyanate. The film can be a conductive material selected from the group consisting of silver, gold, platinum, polyaniline, polypyrrole, poly(3,4-ethylenedioxythiophene). The film can be a metal oxide film. The film can be a piezoelectric material film.

A method for manufacturing of hybrid nanostructured textile sensors (FIG. 2) comprises: feeding one or more polymers and a matrix polymer in molten form through respective extruders to a spinneret to produce fibers having filaments of the one or more polymers in the matrix polymer, the filaments having dimensions of from about 10 to about 100 nanometers; cutting the fibers to a length of from about 0.1 to about 1.5 mm to produce nanofibers; activating the cut nanofibers in a reactor; drying the activated nanofibers; applying an adhesive to a conductive fabric; depositing the activated nanofibers as vertically standing nanofibers, the depositing step including performing an electrostatic and/or pneumatic assisted deposition process using a high strength electrostatic field of 2 kV/cm-10 kV/cm to electrostatically charge the activated nanofibers and deposit the electrostatically charged activated nanofibers as vertically standing nanofibers; curing the conductive fabric containing the vertically standing nanofibers; and electroless plating the vertically standing nanofibers, the electroless plating including dissolving the matrix polymer on the nanofiber surface to expose embedded nanostructures on the filaments, coating the nanofiber surface with a conductive material, and drying the conductive material to form a conductive film on the nanofibers, and annealing the conductive film coated nanofibers.

In a different variation of this method, a single component micro denier yarn of 10-20 μm diameter is used for making microsensors. The method includes cutting of fibers to a length of from about 0.1 to about 1.5 mm; activating the cut microfibers in a reactor; drying the activated microfibers; applying an adhesive to a conductive fabric; depositing the activated microfibers as vertically standing nanofibers, the depositing step including performing an electrostatic and/or pneumatic assisted deposition process using a high strength electrostatic field of 2 kV/cm-10 kV/cm to electrostatically charge the activated microfibers and deposit the electrostatically charged activated microfibers as vertically standing nanofibers; curing the conductive fabric containing the vertically standing microfibers; and electroless plating the vertically standing microfibers, the electroless plating including coating the microfiber surface with a conductive material, and drying the conductive material to form a conductive film on the microfibers, and annealing the conductive film coated microfibers.

A more detailed description of the process can be found in patents U.S. Pat. No. 10,131,993 B2 “Large Scale Manufacturing of Hybrid Nanostructured Textile Sensors,” and U.S. Pat. No. 10,231,623 B2 “Roll-to-roll Printing Process for Manufacturing a Wireless Nanosensor,” the entire disclosures of which are hereby incorporated by reference.

Sensor array functionality: The inter-digitated nanosensor stack with hydrogel can be used as pH probe. All the electrodes are covered with hydrogel, but only the working electrode has the thermosensitive hydrogel combined with salt solution such as potassium chloride. The sensor(s) are able to differentiate between wound surface and skin surface through detecting the pH i.e., normal skin has a pH between 4 and 5.5, a wound has a pH between 7.15 and 8.9, blood has a pH between 7.35 and 7.45.

Thermoelectric coated nanosensors on the inter-digitated electrodes are used to detect change in temperature. Thermoelectric material, such as zinc oxide, is conformally coated on the nanostructured sensor surface to detect change in temperature based on change in electric current between working and counter electrode. Ideally, the dressing should be able to maintain an optimal temperature (from about 98.4° F. to about 99.4° F.) to enhance healing rate. Moisture level is detected by measuring impedance on the wound surface. A current of up to 5 mA at frequency of between about 1100 kHz is passed between a pair of nanostructured electrodes and voltage is measured across another pair of nanostructured electrodes. Electrical potential (AC) from a few micro amps to about 10 mA at frequency range in about 1-100 Hz is passed between a pair of nanostructured electrodes. The current also passes through wound tissue to enhance wound healing rates. This amplitude and frequency of current delivered from the sensor array to the wound will depend on the other metrics measured. Electric potential can intermittently be applied during measurements performed several times during the day. Alternately, depending on the severity/type of the wound, electric potential can be applied to the wound site for longer periods continuously. Silver depletion is measured as change in impedance through nanostructured inter-digitated sensor pair coated with silver nanoparticle-hydrogel layer. These sensors with large surface area which will enhance the measurement capability.

Oxygen content at the wound site is measured by Inter-digitated electrodes and surface acoustic waves in the range of 5 to 100.

Two different formulations of the hydrogel are used with thermo sensitivity of the gel differing by at least 5° C. The hydrogels can be embedded with drugs that have anti-microbial and/or wound healing properties. The hydrogel with lower temperature response releases the drug upon being triggered by body heat when the hydrogel comes in contact with the wound site. Further drug delivery can be triggered by applied heat (created by inter-digitated electrode pair by applying voltage across the electrode). The higher temperature response gel will only be activated if infection is detected (temperature increase, moisture build, abnormal pH, silver depletion using impedance scan).

Biofilms and biofouling formation is prevented as the sensor and electronics module (16) operates in a feedback loop, where the device learns about the current state of the wound and administers the necessary therapeutic modality. This feedback loop operates based on the ingested sensor data that indicates the wound status and bacterial activity. Inducing electrotactic behavior through the application of physiologically safe currents is one possible strategy in continuously keeping the bandage resistant to biofilms throughout the wound healing process. This prevents overall bacterial resistance, thereby preventing biofilms and biofouling. Electric pulse can be used to manipulate bacterial electrotaxis without using chemicals or antimicrobials. While the electric pulse solution decreases bacterial cellular velocity and growth, it does not affect the integrity of normal cells. The amount of current used will be less than 10 mA, which is safe for normal cells and human body. Similarly, electric pulse at wound sites can introduce the release of cytokines and prostaglandins which attracts the macrophages to the site, leading to bacterial death.

### Electronics Module

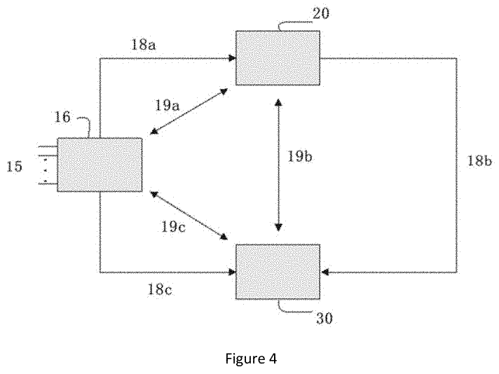

FIG. 4 illustrates data flow of the wound management system. When the electronics module (16) and the phone (20) are powered on, the electronics module and the phone are connected to each other wirelessly. Similarly, the electronics module (16) can be connected via Wi-Fi to the web portal (30). The signal acquisition and transfer (18a-c) are established though command and response mechanism (19a-c) through wireless communication such as Bluetooth, Wi-Fi, or other wireless communication standards. Commands and response (19a-c) between the electronics module, phone, and the web portal are omnidirectional. Examples of such commands and response include, but are not limited to, commands from the smart phone and/or web portal to the electronics module to initializing the acquisition unit, requesting to send data to the phone and/or web portal for signal quality check at the beginning stage of test, requesting start and stop test, and requesting to upload stored data in the storage of the electronics module through wireless communication. Once a test is started, the electronics module acquires and processes signals from sensors. The processed signals are stored to the storage (26) such as a SD card or flash memory. Once a test is completed, the stored data is transferred and uploaded to the phone or server or the portal through wired connection or wireless connection.

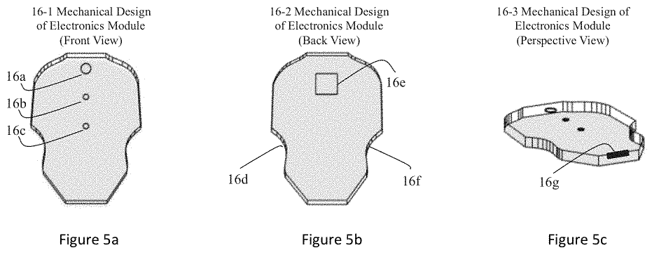

An example of a design of the electronics module is shown in FIG. 5(a)-(c). Power on/off switch (16a), LED indicators and audio-based alarms (16b), (16c) are present in the face of the electronics module as shown in the front view (FIG. 5(a)). The back view (FIG. 5(b)) shows the electromechanical connector (16e) that connects the electronics module (16) to the bandage system (15). The electromechanical connector (16e) can also be used to recharge the power supply (28) present in the electronics module (16). This view also illustrates the ergonomic groves (16d), (16f) for single handed operation. The perspective view (FIG. 5(c) shows the slot (16g) for storage (26).

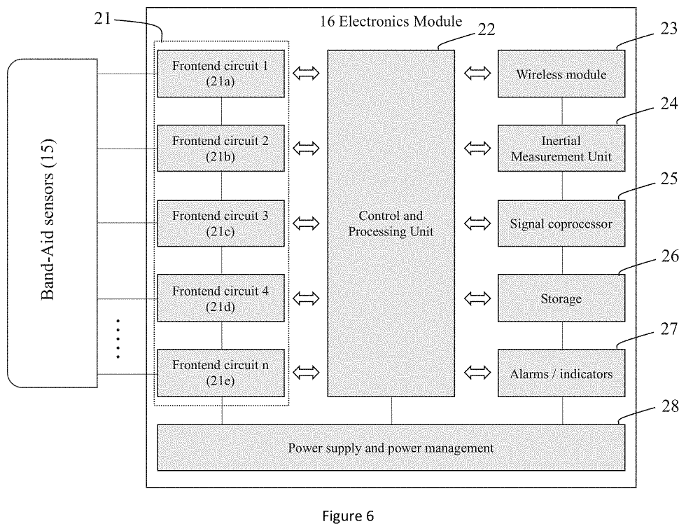

FIG. 6 illustrates the block diagram of the electronics module. The module is designed to be suitable for integration or connection to a bandage including sensors (15). A frontend circuit (21) for impedances can have multiple modulation and demodulation circuits to measure multiple impedance vectors. A frontend circuit for impedances can have multiple modulation and demodulation circuits to measure multiple impedance vectors. The frontend circuit is able to e.g., measure temperature, pH, oxygenation, moisture, depletion or release of any material components of the bandage including the sensors with respect to the wound status. For example, FIG. 6 shows Frontend circuit (21a) for measuring temperature, Frontend circuit (21b) for measuring pH, Frontend circuit (21c) for measuring oxygenation, Frontend circuit (21d) for moisture and Frontend circuit (21e) for measuring depletion or release of any material components.

The electronics module can also supply electric pulse and/or potential to the sensors in the range of 0.001-100 kHz and up to 50 mA. A frontend circuit can have multiple amplifiers, filters, potentiostat circuits to measure a plurality of signals from a combination of multiple sensors. The electronics module has a signal coprocessor unit (16) that is used for signal processing and for additional functions that complement the processing unit. This signal coprocessor unit (25) is connected to the control and processing unit (13) where the executable code is installed. An Inertial Measurement Unit (24) is connected to the control and processing unit (22) for movement tracking. The electronics module also has indicators and/or alarms (27), for status updates. The power supply and management block (28) of the electronics module provides the proper voltages and power to each circuit and the sensors from a battery to power up the electronics module. A wireless module (23) enables the electronics module (16) to communicate with the smart phone and web portal. Storage (26) such as a SD card or flash memory is used to store data.

### Bandage

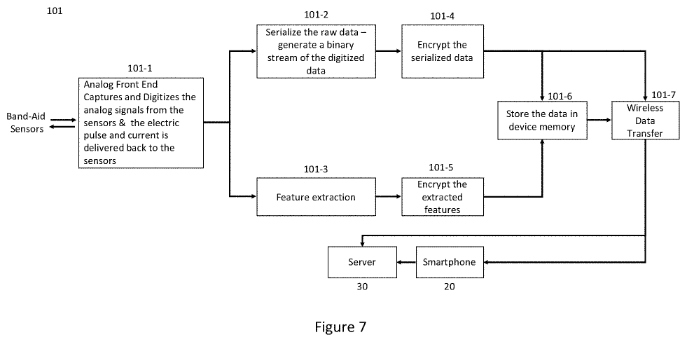

As shown in FIG. 7, the electronics module has a control and processing unit (22) that is a microprocessor or microcontroller with executable code 101 that performs the functions of capture and conversion of the signals from the bandage into machine readable digitized data. Created arrays of data can be organized in a traditional file system for subsequent retrieval in a local storage medium. This storage medium is non-volatile memory that can be erased and programmed as needed. The code 101 also communicates to an internet connected database service that resides in a remote physical database server. It can communicate with the server directly and transfer the acquired data for a patient as needed. The control and processing block can capture and digitize data from several channels that can have different sampling frequency requirements. The code 101 can serialize the data acquired and generate packets on a per second basis or at a frequency equivalent to the lowest sampling frequency.

FIG. 7 shows illustrative steps of code 101, in further detail. Code 101 preferably captures and digitizes data from several channels that can have different sampling frequency requirements (step 101-1). In this regard, the code 101 can serialize the digitized data and generate packets on a per second basis or at a frequency equivalent to the lowest sampling frequency (step 101-2 and 101-3). Code 101 also performs feature extraction (step 101-3). These extracted features are then encrypted (101-4 and 101-5) and stored (101-6) in memory (17), for later transmission to the smart phone (20) and/or web server (30) (101-7). It should be noted that although the feature extraction is preferably performed on the electronics module (10), it is also possible to instead perform these steps on smart phone (20) or web server (30).

### Software Application

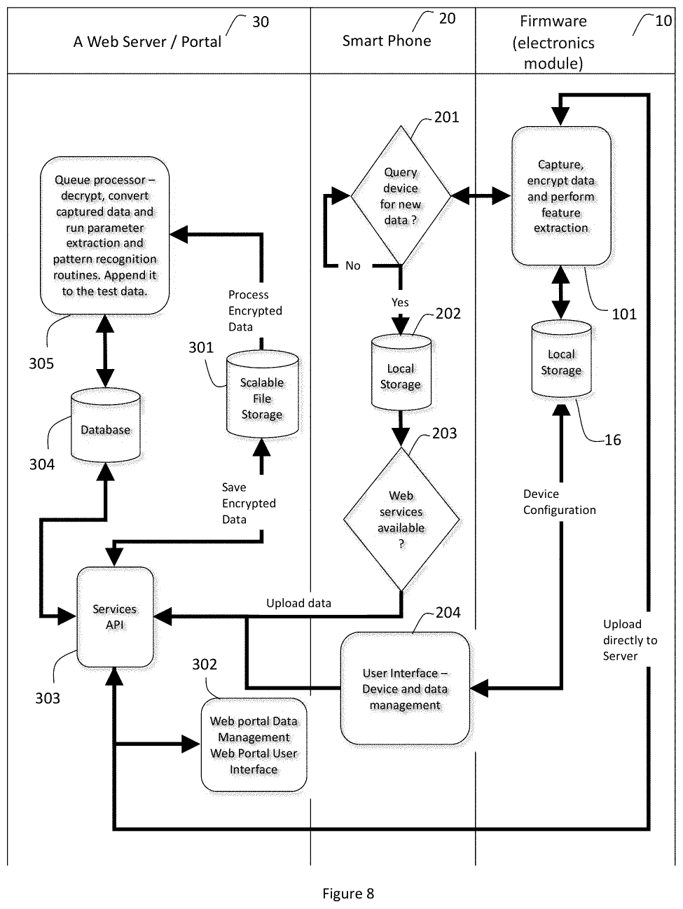

The smart phone (20) includes a microcontroller or microprocessor and a storage medium that contains microcontroller or microprocessor executable code 201. The code 201 can perform the functions related to the command and interface functions shown in FIG. 8. The command responses may include but not limited to the following list of commands and their associated responses and descriptions:

| Command Name | Command code | Command Description |
|---|---|---|
| ACC_FileDuration | 0x00 | This command is used to set the amount of |
| | | data to be stored in a single file. |
| ACC_StatusVariable | 0x01 | This command is used to query the module for |
| | | its current status. |
| ACC_StartAcquisitionRequest | 0x02 | This command will start the signal data |
| | | acquisition from the Analog front End. The |
| | | acquired data is encrypted and stored on the |
| | | SD card. |
| ACC_RealTimeClockSet | 0x03 | This command is used to set the Real Time |
| | | Clock and Calendar values on the |
| | | Microcontroller. This operation needs to be |
| | | done to synchronize the time between the |
| | | smart device 20 and the time included in the |
| | | data files that are captured by 10. |
| ACC_FileTransferRequest | 0x04 | This command is used to request completed |
| | | files from the module. The module will search |
| | | for the requested file and start transferring the |
| | | contents of the file from the Signal acquisition |
| | | unit |
| ACC_RealTimeReadRequest | 0x05 | This command returns the value of the Real- |
| | | time clock and calendar on the Electronics |
| | | module 10. |
| ACC_StopAcquisitionSleep | 0x06 | This command instructs the module to go to |
| | | sleep mode where all functions of the |
| | | Electronics module are suspended. |
| ACC_RealTimeStreamRequest* | 0x07 | Streams the acquired data to the smart device |
| | | in a specified format |
| ACC_SetEncryptionKey | 0x08 | This command is used to set the encryption |
| | | key on the Electronics module at the beginning |
| | | of the test. |
| ACC_CurrentFileNumber | 0x09 | This command is used to query the module to |
| | | determine the current file number. |
| ACC_DeviceID | 0x0A | This command is read the device ID |
| | | information |
| ACC_FileSize | 0x0B | This command is used to determine the |
| | | number of bytes in each file. |
| ACC_FileTransferCancel | 0x0C | This command is used to cancel an ongoing |
| | | file transfer. |
| ACC_SendKeepAlive | 0x0D | This command is used to request the |
| | | Electronics module to send message beacons |
| | | that serve as pings to keep the smart device |
| | | app active. |
| ACC_DataSession | 0x0E | This command is used by the Smart device to |
| | | inform the Electronics module that a data |
| | | connection is either started or is about to |
| | | terminate. |
| ACC_GetGPSlocation | 0x0F | This command is used by the Electronics |
| | | module to inquire the smart device and |
| | | retrieve GPS location including altitude |
| | | from a GPS service on the smart device. |

The code in 201 has access to local storage available in the smart device 202. The code in 201 can further inquire the operating system in smart device (20) regarding the availability of an internet connection that will allow communication to web services 203. The code in 201 further communicates with a user interface and data managing software module 204. The interfaces between the patient or end user and the smart device are implemented by this module.

The Web server/portal (30) is implemented as two services that work in tandem, in an asynchronous manner. The services Application Programming Interface (API) 303 are responsible for collecting the data acquired by the Electronics module (10), received either directly from the Electronics module 101 or through the smart device software module 201. The API 303 routes the data to a secure cloud storage database that is capable of auto-scaling 301 to meet increased demands as needed. As soon as new data files are available in Scalable File Storage 301, a web server app hosted in 305 processes the data files. The processing in 305 may include, but is not limited to the following:
1) Decryption
2) Parsing of the raw data to separate them into individual channels of physiological data
3) Data type and format conversions
4) Calculation/extraction/pattern recognition tasks that allow the determination of parameters/features and a predictive score about wound healing.
5) Derive wound healing status and rate based on the original data measured by the system or any feature extracted by the step 4 above. To draw conclusions on the patient or end user's current wound status, prognosis, treatment recommendations, and predictions about wound healing using artificial intelligence, machine learning techniques, and computational statistical modeling techniques including, but not limited to, heuristics, support vector machines, neural networks and artificial neural networks, and Markov decision process, unsupervised learning, supervised learning, reinforcement learning, decision trees, regression analysis, self-learning, Bayesian networks, odds ratio, constant false alarm rate, weighted sum, threshold metrics, random forest, and fuzzy logic/neural network.

After the data files have been processed by 305, the resulting meta-data, features or parameters are stored in database 304. The databases 301 and 304 may be combined in a single database in a manner that is known to any person skilled in database management systems.

The web portal front end 302 is responsible for the management of the processed data and generating a user interface wherein the data is presented in a human readable form to a physician. The web portal front end 302 accesses the data that has been processed by 305 through the database services in 303. Apart from receiving the data collected by the device, the web portal can also ingest data including, but not limited to height of the patient, a weight of the patient, a gender of the patient, an age of the patient, a medical history and physical examination records of the patient, a medical status of the patient, a body mass index (BMI) of the patient, an ethnicity of the patient, a medical prescription history of the patient, a medical prescription status of the patient, types of medical treatments for the wound received by the patient, types of medical treatments for health issues and insurance or claims information previously received by the patient, diet information for the patient, psychological history of the patient, and a genetic indicator of the patient, biomarkers of the patient along with other EMR information.

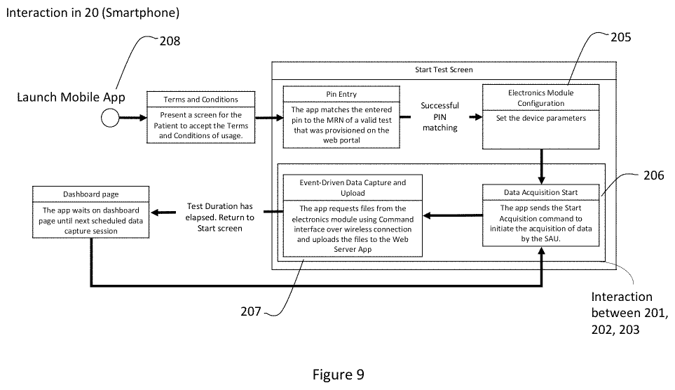

The overall operation of the Smartphone Mobile App (FIG. 9) involves four key states. The four states are idle or initial app launch state, configuration of the electronics module, starting a test or recording and event-driven transfer of data from electronics module to the smart device and the subsequent upload of the transferred data. FIG. 9 shows the sequential commands of the Launch Mobile App 208 sent to the Electronics Module Configuration 205 to configure it before the start of a test or recording, sequential commands sent from the Mobile App to the Electronics module in order to start the Data Acquisition Start 206 for data acquisition for a test, and sequential commands sent from the Smartphone App to the Electronics Module when the App is restarted after being suspended or shutdown respectively. The Event Driven Data Capture and Upload 207 is the App that requests files from the electronics module using Command interface over wireless connection and uploads the files to the Web Server App. The data collection operation is entered under the following assumptions on the state of the test:
The Electronics module and the Smartphone have been paired via Bluetooth
The Smartphone software application has been installed on the smart device.
The test initiation steps on both the Web Server/portal and the Mobile App have been completed to the Start Acquisition phase

The data collection process is event-driven and triggered by the electronics module with two messages send to the smart device:
The ACC_CurrentFileNumber message sent by the device when it starts collecting a new file of data.
The electronics module also generates a request to the smart device to launch the application when an active communication session has not been established

The Data flow diagram depicts the state machine that implements the event driven querying operation between the Smartphone App and the electronics module, and between the smartphone app the Web Server/portal.

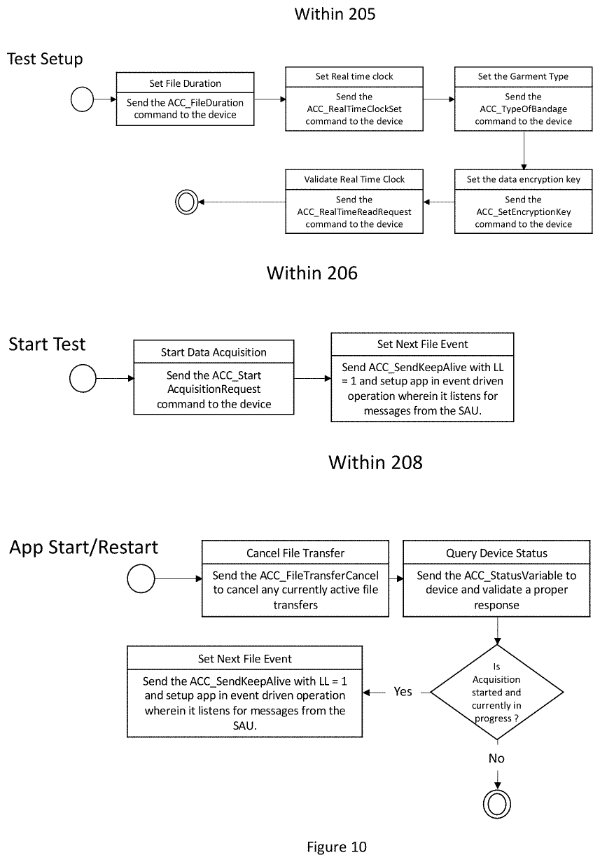

FIG. 10 describes the interaction within Electronics Module Configuration 205, Data Acquisition Start 206, and Launch Mobile App 208.

More specifically 205 relates to the test or recording setup process that takes place in the electronics module. The steps include:
Setting the file duration, which is used to set the amount of data to be stored in a single file.
Setting real time clock, which is used to set the Real Time Clock and Calendar values on the Microcontroller. This operation needs to be done to synchronize the time between the smart device (20) and the time included in the data files that are captured by the Electronics Module (10).
Setting the garment type, which is used to determine the type of bandage that is used for a recording if there are multiple options of bandage available.
Setting the encryption key, which is used to set the encryption key on the Electronics Module at the beginning of the test.
Validating the real time clock, which is used to validate the real time clock values set during the beginning of the recording.

Similarly, (206) relates to the test initiation process that take place in the Electronics Module and the App. The steps include:
Data acquisition, which is used to start the signal data acquisition from the Analog front End. The acquired data is encrypted and stored on the SD card in the electronics module.
Setting next file event, which is used to set up the App for event driven operations wherein it listens for the messages from the Electronics Module.

Likewise, (208) relates to starting/restarting the App. The steps include:
Stopping file transfer, which is used to cancel an ongoing file transfer.
Querying device status, which is used to determine the operational status of the device.
Setting next file event, which is used to set up the App for event driven operations wherein it listens for the messages from the electronics module.

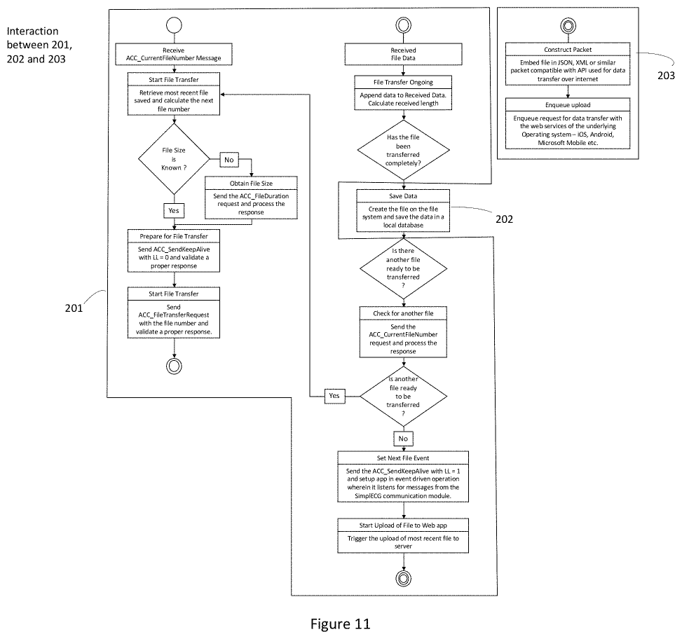

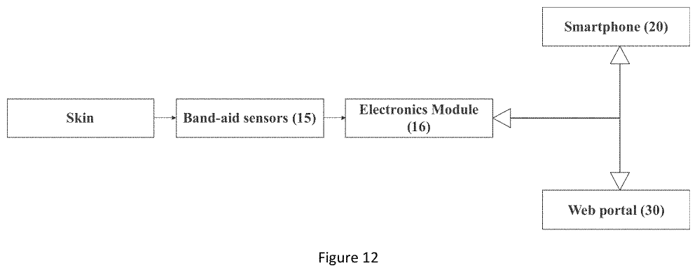

As shown in FIG. 11, using the commands in the table in the software section, the code 201 sends and retrieves data from the electronics module. The commands were created following a standard programming design pattern known as command pattern known to those skilled in the art. The code 201 also stores and accesses data on the local storage 202 in the smart phone (20), including, for example, storing data received from the electronics module. Code 201 can further communicate with the operating system code 203 in smart phone (20) regarding the availability of an internet connection that will allow communication to web services, and codes 201, 203 effect uploading of data to the web server (30). The code in 201 further communicates with a user interface and data managing software module 204. The interfaces between the patient or end user and the smart phone are implemented by this module. The overall wound management system deployment is shown in FIG. 12. The deployment of the Bandage sensors (15) on the skin shown. The electronics module (16) is connected to the Bandage sensors (15). The electronics module (16) communicates wirelessly with a smartphone (20) and/or with the web portal (30).

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

Obvious variants of the disclosed embodiments are within the scope of the description and the claims that follow.

All references cited herein, as well as text appearing in the figures and tables, are hereby incorporated by reference in their entirety for all purposes to the same extent as if each were so individually denoted.

What is claimed is:

1. A Band-Aid system for treating and detecting a plurality of physiological values from the wound or wound site with sensors, transducers, and connectors.

2. The wound management system of claim 1, wherein the hydrogel is functionalized with therapeutic chemical compounds selected from the group consisting of antiseptic agents, microbial agents, buffer stabilized moisture (or saline), antibiotics, antibodies and combinations thereof.

3. The system of claim 1, wherein at least one of the sensors is used to acquire and measure pH value from the wound site.

4. The system of claim 1, wherein at least one of the sensors is used to acquire and measure temperature of the wound site.

5. The system of claim 1, wherein at least one of the sensors is used to acquire and measure electrical impedance of the wound site.

6. The system of claim 1, wherein at least one of the sensors is used to acquire and measure moisture at the wound site.

7. The system of claim 1, wherein at least one of the sensors is used to supply an electric potential to the wound site.

8. The system of claim 1, wherein at least one of the sensors is used to acquire and measure the bacterial activity at the wound site.

9. The system of claim 1, wherein at least one of the sensors is used to acquire and measure the bacterial activity or supply an electric potential is used to prevent biofilms and biofouling.

10. The system of claim 1, wherein at least one of the sensors has a combination of comprising a working electrode (WE), a counter electrode (CE), and a silver-silver chloride reference electrode (RE).

11. The system of claim 1, wherein at least one sensor is coated with hydrogel and thermosensitive hydrogel-drug or buffer formulation

12. The system of claim 1, wherein at least one of the sensors is a nanosensor.

13. The system of claim 1, wherein at least one of the sensors is an interdigitated electrode or transducer.

14. The system of claim 11, wherein the nanosensors have vertical or helical nanostructures.

15. The system of claim 12, wherein the nanostructures are polyethylene terephthalate, polyethylene naphthalate, polybutylene terephthalate, polyester, polyurethane, polystyrene, polyvinyl alcohol, ethylene vinyl alcohol, polyacrylamide, poly lactic acid, or polyethylene terephthalate.

16. The system of claim 1, wherein the sensors, detectors, or transducers are connected to a connector that establishes electrical connection to the electronics module.

17. A Band-Aid system and electronics system for assessing wound status information and rate of healing of the wound comprising a Band-Aid connected to the electronics module.

18. The system of claim 16, wherein the electronics module is connected to a connector that establishes electrical connection to the electronics module.

19. The system of claim 16, wherein the electronics module acquires, processes, stores, and transfers the sensor data.

20. The system of claim 16, wherein the electronics module generates electric potential to be supplied to at least one of the sensors in the Band-Aid.

21. The system of claim 16, wherein the electronics module comprises an Inertial Measurement Unit.

22. The system of claim 16, wherein the electronics module comprises a signal coprocessor.

23. The system of claim 16, wherein the electronics module microprocessor or microcontroller with executable code that performs the functions of capture and conversion of the signals from the Band-Aid into machine readable digitized data.

24. The system of claim 16, wherein the electronics module comprises indicators and/or alarms for status updates

25. The system of claim 16, wherein the electronics module comprises a wireless module to communicate or transfer data with the smart phone and web portal at a remote location.

26. A system for predicting wound healing and providing treatment guidelines: a Band-Aid with plurality of sensors, an electronics module to acquire and process the data from Band-Aid, and store and/or transmit the data; a software application installed on a phone and/or web portal that receives the data from the electronics module for further data analysis.

27. The system of claim 25, wherein system performs the functions of decryption, parsing of the raw data to separate them into individual channels of physiological data, data type and format conversions, calculation/extraction/pattern recognition tasks that allow the determination of parameters/features, perform pattern recognition tasks on any of the extracted features, to draw conclusions on the patient or end user's current wound status, prognosis and treatment recommendations.

28. The system of claim 25, wherein system generates a report about the status of the wound.

29. The system of claim 25, wherein the system provides an assessment about the wound and provides a treatment guideline based on the assessment.

30. The system of claim 25, wherein the system can autonomously impart treatment changes based on the data from the sensors, detectors, transducers, and electronics module.

31. The system of claim 25, wherein the software application can be used to impart changes in treatment.

32. The system of claim 25, wherein the system performs analysis to predict the wound status and healing rates at a specific point in time based on the specific patient data and on historical data regarding outcomes of a plurality of wound healing rates on a plurality of similar patients.

33. The system of claim 25, wherein the system performs analysis to predict the outcome of the change in treatment for a patient based on the baseline patient data and previously computed healing rates.

34. The system of claim 25, wherein the system predicts a degree of a confidence for each of the predicted outcomes indicating a chance that the patient will achieve the predicted outcome associated therewith at the specific point in time based on the treatment, each of the degrees of confidence based at least on the patient data and on the historical data regarding outcomes of the plurality of wound healing rates on the plurality of patients.

35. A bandage system for detecting physiological parameter and/or biomarker values from a wound site comprising a bandage, said bandage having:
sensors for detecting one or more physiological parameters,
a thermosensitive hydrogel,
an adhesive for securing the bandage to skin,
a connector which can connect the bandage to an electronics module for processing of the values electrical connection lines capable of relaying signals from the sensors to the connector.

36. The bandage system of claim 1, wherein the electronics module is integrated or connected to the bandage and wherein the electronics module is comprised of a frontend circuit to measure signals, a signal coprocessor unit for signal processing which is connected to a control and processing unit for converting signals from the sensors to computer readable data, a power supply and management block to provide proper voltages and power to the frontend circuit and the sensors and a wireless module to enable the electronics module to communicate the data to a computer.

37. The bandage system of claim 1, wherein the hydrogel is functionalized with therapeutic chemical compounds selected from the group consisting of antiseptic agents, microbial agents, buffer stabilized moisture (or saline), antibiotics, antibodies, antibiofilm agents and combinations thereof.

38. The bandage system of claim 1, wherein the nanosensor is comprised of a nanostructured working electrode (WE) and counter electrode (CE), and silver-silver chloride reference electrode (RE).

39. The bandage system of claim 3, wherein the bandage further includes a heating element connected to the layer of electrical connection lines for heating the skin and for activating release of the therapeutic chemical compound from the hydrogel.

40. The bandage wound of claim 1, wherein the physiological parameters are selected from the group consisting of oxygen, silver depletion, pH, temperature, moisture level, bacterial activity and combinations thereof.

41. The bandage system of claim 1, wherein at least one of the sensors is used to supply an electric potential to the wound site.

42. The bandage system of claim 1, wherein at least one sensor is coated with hydrogel and a thermosensitive hydrogel-drug or buffer formulation

43. The bandage system of claim 1, wherein at least one of the sensors is a nanosensor.

44. The bandage system of claim 1, wherein at least one of the sensors is an interdigitated electrode.

45. The bandage system of claim 9, wherein the nanosensors have vertical or helical nanostructures.

46. The bandage system of claim 9, wherein the nanostructures are polyethylene terephthalate, polyethylene naphthalate, polybutylene terephthalate, polyester, polyurethane, polystyrene, polyvinyl alcohol, ethylene vinyl alcohol, polyacrylamide, poly lactic acid, or polyethylene terephthalate.

47. A bandage and electronics system for assessing wound status information and rate of healing of a wound comprising the bandage system of claim 1 connected to an electronics module.

48. The bandage and electronics system of claim 13, wherein the electronics module acquires, processes, stores, and transfers the data obtained by the sensors.

49. The bandage and electronics system of claim 13, wherein the electronics module generates electric potential to be supplied to at least one of the sensors.

50. The bandage and electronics system of claim 13, wherein the electronics module includes an inertial measurement unit, a signal coprocessor, a microprocessor or microcontroller with executable code for performing the functions of capture and conversion of the signals from the sensors into machine readable digitized data, indicators and/or alarms for providing status updates, and a wireless module capable of communicating or transferring data to a smart phone and/or a web portal at a remote location.

51. A system for detecting and processing data from a wound and providing treatment guidelines for healing of the wound comprising:
a bandage having a plurality of sensors;
an electronics module capable of acquiring and processing the data from bandage and storing and/or transmitting the data;
a software application installed on a phone and/or web portal for receiving the data from the electronics module for analysis of the data and for providing treatment guidelines for healing of the wound based on the results of the analysis.

52. A method of detecting and processing data from a wound and providing treatment guidelines for healing of the wound using the system of claim 17, wherein the system performs the functions of decryption, parsing of the data obtained from the sensors to separate the data into individual channels of physiological data, data type and format conversions, calculation/extraction/pattern recognition tasks that allow the determination of parameters/features, performing of pattern recognition tasks on extracted features to draw conclusions on the current status of the wound, and providing evaluation of prognosis and treatment recommendations based on the conclusions.

53. The method of claim 18, wherein the system autonomously imparts treatment changes based on the data from the sensors, and the electronics module.

54. The method of claim 18, wherein the system analyzes the data from the sensors, baseline patient data and previously computed healing data to provide treatment recommendations for the wound.

55. A method for managing wounds comprising applying a bandage containing a sensor to a wound site to measure one or more physiological values:
measuring the one or more biomarker and/or physiological values,
sending the measured physiological values from the sensor to an electronics module via an electromechanical connector,
translating the measured biomarker and/or physiological values into machine readable data,
transmitting the computer readable code to a computer;
comparing the biomarker and/or physiological values from the sensor to predetermined physiological values stored in the computer to obtain a comparison analysis in real time,
providing a management and treatment plan for the wound based on the results of the analysis.

56. The method for managing wounds of claim 21, wherein the measured physiological values are selected from the group consisting of oxygen, silver depletion, pH, temperature, electrical impedance, moisture level, bacterial activity and combinations thereof.

57. The method for managing wounds of claim 22, wherein the treatment plan includes a determination of the frequency and magnitude of delivery of therapeutics for treating the wound.

58. The bandage wound management system of claim 9, wherein the nanosensor is a hydrogel stack cloth based nanosensor array located on the bandage

59. The bandage wound management system of claim 24, wherein the stack has the ability to inject electrical pulse of varied potential and frequency into the wound.
